(19) United States
(12) Patent Application Publication
(10) Pub. No.: US 2026/0207942 A1
(43) Pub. Date: Jul. 23, 2026

(54) DEVICE AND SYSTEM FOR ELECTRICALLY STIMULATING THE EYE

(71) Applicant: Okuvision GmbH, Reutlingen (DE)

(72) Inventors: Steffen Berenbold, Horben (DE); Nicolò Ceretto Gianone, Torino (IT); Paul Henzler, Nuertingen (DE); Dorothee Stoelzel, Rottenburg (DE); Fabian Kaiser, Pfullingen (DE)

(73) Assignee: Okuvision GmbH, Reutlingen (DE)

(21) Appl. No.: 19/569,836

(22) Filed: Mar. 17, 2026

Related U.S. Application Data
(63) Continuation of application No. PCT/EP2024/075868, filed on Sep. 17, 2024.

(30) Foreign Application Priority Data
Sep. 18, 2023 (DE) 10 2023 125 136.8

(51) Int. Cl. A61N 1/36; A61N 1/04

(52) U.S. Cl. CPC A61N 1/36046; A61N 1/0408; A61N 1/0472

(57) ABSTRACT

A device for electrostimulation of an eye includes a spectacle-like frame with a nose part. An arrangement is connected to the nose part for holding the spectacle-like frame on a head of a user. At least one holder is arranged at the spectacle-like frame for an element to be brought into engagement with the eye of the user. The element is a wire-shaped stimulation electrode, and the at least one holder is adapted for back and forth movement between a non-engagement position and a predefined engagement position.

### CROSS-REFERENCE TO RELATED APPLICATION

This application is a continuation of International Patent Application No. PCT/EP2024/075868, filed on Sep. 17, 2024, which claims priority to German Patent Application No. 10 2023 125 136.8, filed on Sep. 18, 2023, the disclosures of which are hereby incorporated herein by reference in their entireties.

### TECHNICAL FIELD

The present disclosure relates to a device, in particular for electrostimulation of the eye, and a system for electrostimulation of the eye, in particular for transcorneal electrostimulation, wherein the device comprises a spectacle-like frame with a nose part, an arrangement connected to the nose part for holding the frame on the head of the user, and at least one holder arranged at the frame for an element to be brought into engagement with an eye of the user, in particular for a wire-shaped stimulation electrode.

### BACKGROUND

WO 2011/086150 A2 generally discloses stimulation electrodes, electrode holders, and devices for electrostimulation.

The known stimulation electrode is a wire-shaped electrode which is attached to the spectacle-like frame. When the frame is being arranged on the head of a patient, the flexible wire electrode comes during putting on the spectacle-like frame into contact with the front surface of the eye in the lower edge region of the cornea of the eye. A counter electrode is either provided on the frame or is attached separately at the patient, for example on forehead of the head or on an earlobe.

The wire electrode and the counter electrode can be used to route a pulse-like electrostimulation signal into the eye, leading to a stimulation current flowing via the cornea into the eye.

The known device and other devices are used to route electrostimulation signals into the eye because it was found that this can stabilize or even improve certain forms of retinopathia pigmentosa and other eye diseases.

Retinal degeneration is a significant cause of blindness in industrialized countries. Various trials provide indications that low electrical currents which flow through the retina can delay the onset of genetic, age-related or sudden degeneration of the retina.

This opens up the possibility of being able to delay the gradual loss of sight through of regular, that is to say e.g. daily or weekly, electrostimulation of the eye in order thus to maintain parts of the vision for longer in affected patients, or even restore parts of the vision.

Expediently, this treatment should be carried out by the patient himself and at home. In doing so, in the extreme case, the assumption must be made that the patient is practically blind. The result of this is that there are significant demands on the device and the stimulation electrode mentioned at the outset.

This is because the wire-shaped stimulation electrode must be replaced after every use, or at least very frequently. On the one hand, the wire-shaped stimulation electrode is a very thin electrode, which can break in the case of improper or even frequent use.

On the other hand, considerations in respect of hygiene may make it necessary to replace the stimulation electrode after each use.

WO 2011/086150 A2, mentioned at the outset, describes three different embodiments for these applications.

In a first embodiment, the device for electrostimulation of the eye is designed in the style of spectacles, with spiral electrode holders, which extend towards the eye, thereby being provided on the frame which is usually provided for the lenses. Two such electrode holders are provided for each eye, and so a wire electrode needs to be connected to the two inner ends of these flexible electrode holders in order to come into contact with the cornea when the spectacles are put on.

In a second embodiment, a facemask is shown, which is fitted over an area to parts of the face of the patient. Eye openings, on which pushbuttons are provided, into which the wire-shaped stimulation electrodes are hooked, are provided in this facemask.

In a third embodiment, a type of “shooting spectacles” is shown, in which the spectacles-like frame comprises a nose clip and two eye brackets coming off the former, which eye brackets are in turn connected to foldable earpieces.

Each two electrode holders are attached to the eye brackets; said electrode holders can be adjusted along the eye bracket and perpendicular to the eye bracket, i.e., in all three directions.

On their inner free ends, the electrode holders are respectively connected to ears, into which the wire-shaped stimulation electrode is introduced. The stimulation electrode is attached with its first end to the nose-side electrode holder and is then routed through an ear to the temple-side electrode holder. At its second end, the stimulation electrode is weighed down by a weight such that the stimulation electrode is pretensioned in this manner.

A person skilled in the art fits this frame to the head of the patient such that, when such spectacles which are equipped with one or two stimulation electrodes are put on, the stimulation electrode automatically rests against the eye. A further device that is intended to enable flexible adjustment with many degrees of freedom is described in WO 2015/104313 A1. Upon one-time adjustment, the device is firmly fixed with a clamping screw as a fixing device.

While a person skilled in the art sets the geometric conditions of the frame on one occasion, the patient should subsequently use the device at home in order to subject his eyes to regular electrostimulation.

However, hooking the stimulation electrode into the ears or onto the electrode holder, which is required for this, is found to be extremely difficult for patients with visual impairments. In part, the thin electrode thread is not recognized by the patients or said electrode thread can slide out of the electrode holders when the spectacles frame is put on.

In order to prevent this, the stimulation electrode must be wound a number of times around the holder at the nose-side electrode holder. This requires much dexterity because, firstly, the thread is rigid and, secondly, the patients have to resort to their sense of touch as a result of their visual impairment.

Against this background, DE 10 2011 055 844 B4 discloses a further improved stimulation electrode arrangement for a device for electrostimulation of the eye. The device comprises a spectacle-like frame with a nose part, an arrangement connected to the nose part for holding the frame on the head of the patient, and at least one electrode holder, which is arranged on the nose part in adjustable fashion, for the wire-shaped stimulation electrode. The stimulation electrode arrangement comprises an electrode carrier, which can be attached to the electrode holder in detachable fashion and on which the stimulation electrode is arranged. In an embodiment, the electrode carrier is formed as a U-shaped bracket with two arms and a groove open to the outside. The solution described in DE 10 2011 055 844 B4 provides a stimulation electrode arrangement of the type mentioned above, which is easy to handle and also facilitates handling for visually impaired patients.

In the solution described in DE 10 2011 055 844 B4, the patient shall only need to grip the stimulation electrode arrangement by the electrode carrier, which is much more massive than the thin stimulation electrode, and attach this electrode carrier in the electrode holder which is already present and adjusted on the device.

Since the electrode carrier has a greater mass and greater dimensions than the stimulation electrode itself, visually impaired or even blind patients can feel the electrode carrier and insert it, in the correct position, into the electrode holder.

In view of the above, it is an object of the present disclosure to provide a device, in particular for electrostimulation of the eye, which further improves handling and enables a more comfortable user experience. In particular, it would be desirable to facilitate the application of an element to be brought into engagement with the eye of the user.

### SUMMARY

According to a first aspect of the present disclosure, it is therefore proposed to provide a device, in particular a device for electrostimulation of the eye, wherein the device comprises a spectacle-like frame with a nose part, an arrangement connected to the nose part for holding the frame on the head of the user, and at least one holder arranged at the frame for an element to be brought into engagement with an eye of the user, in particular for a wire-shaped stimulation electrode, wherein the holder is adapted to be moved back and forth (relative to the frame) between a non-engagement position and a predefined engagement position.

Conventional devices for electrostimulation of the eye usually require two people to apply the spectacles equipped with the stimulation electrodes to the eyes. While an assistant places the spectacles with the stimulation electrodes on the user and simultaneously, during putting on the spectacles, applies the stimulation electrodes on the eyes, the user holds both eyes open with their hands and ensures that the stimulation electrodes do not collide with the eyelashes.

When putting on the spectacles of a conventional electrostimulation device, in particular when the earpieces of the spectacles are threaded or inserted behind the user's ears, a kind of slight sawing motion can occur, i.e., a movement in which the stimulation electrodes are pulled across the eyes of the users in the longitudinal direction of the stimulation electrodes, which can be perceived as uncomfortable by users.

It is therefore proposed that the element to be brought into engagement with the eye of the user, such as a stimulation electrode or contact lens, should not be provided in a rigidly fixed position relative to the spectacle-like frame of the device for the user. Instead, it is proposed to provide a holder arranged at the frame, wherein the holder is adapted to be moved back and forth relative to the frame between a non-engagement position and a predefined engagement position.

As described at the outset, no holder is provided in conventional electrostimulation devices that is adapted accordingly. At most, a one-time adjustment is made to the user's anatomy. The adjustment is fixed, for example, by an ophthalmologist. In subsequent applications, the pre-fixed device with attached electrode holders is put on by the user or an assistant, i.e., the electrodes come into contact with the cornea during placing or putting on the spectacles.

In contrast, the proposed device can be put on first, wherein the holder still is in the non-engagement position. In the non-engagement position, however, the element to be brought into engagement with the eye of the user is not yet in contact or engagement with the eye of the user, even when the device is worn in place. Only after placing or putting on is the holder moved from the non-engagement position to the predefined engagement position. In the predefined engagement position, when the device is worn the element to be brought into engagement with the eye of the user is in engagement or contact with the eye of the user in a predefined position. Instead of in a random manner making contact with the eye of the user, the engagement position of the device is predefined and the holder is specifically adapted to be moved to this predefined engagement position.

The predefined engagement position can be adjusted, for example, by an expert, such as an ophthalmologist. For example, a stop or end stop can be set at which the holder can rest or abut against in the predefined engagement position. For subsequent application, the user can move the holder back and forth between the non-engagement position for placing or putting on the device and the predefined engagement position. In other words, a predefined engagement position within the meaning of the present disclosure can refer to a predetermined or definable or presetable engagement position. In particular, the device can comprise an, in particular adjustable, stop (a limiting device), which is adapted to limit the movement of the holder in the predefined engagement position in a spatial direction. However, it is also possible that the engagement position can be adjusted by adjusting one or more elements of the holder, which in the engagement position can abut against a non-adjustable predetermined stop. The device can be adapted to limit or prevent movement of the holder beyond the predefined engagement position in the direction of the eye in the predefined engagement position. However, the device is still adapted to enable movement of the holder between the non-engagement position and the predefined engagement position. An advantage of this solution can be that it further improves handling for visually impaired users and can enable a more comfortable user experience.

Since the device is adapted to provide a predefined engagement position and since the device is adapted to not simply be moved arbitrarily, but is adapted to be moved back and forth between a non-engagement position and the predefined engagement position, a reproducible positioning of the element to be brought into engagement with the eye of the user, in particular a stimulation electrode on the eye surface, is possible in the engagement position. In particular, reproducible positioning by an untrained user is also made possible.

The device can be a device for electrostimulation of the eye. The holder can be an electrode holder for a (wire-shaped) stimulation electrode or stimulation electrode arrangement. The stimulation electrode arrangement can comprise a first electrode carrier and a second electrode carrier, wherein the stimulation electrode is arranged between the first electrode carrier and the second electrode carrier, wherein the first and/or second electrode carrier is adapted to be magnetically attached to the electrode holder.

The solutions described in their own earlier applications mentioned at the outset already significantly improve the handling of a stimulation electrode for electrostimulation of the eye for visually impaired users. However, the precise placement of the stimulation electrode arrangement on the electrode holder may continue to cause difficulties for visually impaired users. According to this aspect, it is therefore proposed that the stimulation electrode arrangement comprises a first electrode carrier and a second electrode carrier, wherein the stimulation electrode is arranged between the first electrode carrier and the second electrode carrier, wherein the first and/or second electrode carrier is adapted to be magnetically attached to the electrode holder.

By magnetically attaching the electrode carrier to the electrode holder, handling is further facilitated for visually impaired users. The electrode carrier and electrode holder attract each other magnetically, allowing a certain degree of self-positioning of the electrode carrier and electrode holder relative to each other. Approximate or imperfect positioning of the electrode holder, as it can also be achieved by a visually impaired user, can therefore be sufficient to magnetically receive the electrode carrier.

A further advantage of this embodiment can be that hygiene can be improved when handling a stimulation electrode arrangement for a device for electrostimulation of the eye. Due to the electrode carrier adapted to be magnetically attached to the electrode holder, fewer attempts by the visually impaired user to correctly attach the electrode carrier to the electrode holder may be required. This reduces the risk of contamination being inserted into the eye due to improper handling and further improves hygiene during handling.

In the context of the present disclosure, a stimulation electrode arrangement can refer to an electrode assembly for stimulating and/or measuring. Accordingly, a stimulation electrode can refer to an electrode for stimulating and/or measuring. In other words, not only can an electrostimulation signal be delivered, but a measurement signal can also be recorded. For example, with the stimulation electrode or stimulation electrode arrangement, a stimulus can be applied or an electroretinogram or ERG signal can be acquired. Accordingly, in the context of the present disclosure, a device for electrostimulation of the eye can refer to a device for stimulating and/or measuring. Accordingly, in the context of the present disclosure, a system for electrostimulation of the eye can refer to a system for stimulating and/or measuring.

The device can comprise two separate holders, a left holder for an element to be brought into engagement with a left eye of the user, in particular for a stimulation electrode for the left eye, and a right holder for an element to be brought into engagement with a right eye of the user, in particular for a stimulation electrode for the right eye. An advantage can be that handling is further improved.

In a further refinement, the left holder and the right holder can be adapted to be moved separately from each other back and forth between a respective non-engagement position and a respective engagement position. In other words, the left holder can be adapted to be moved back and forth independently of the right holder between a non-engagement position for the left holder and an engagement position for the left holder, in which an element to be brought into engagement with the user's left eye can be brought into engagement with the user's left eye. Accordingly, the right holder can be adapted to be moved back and forth independently of the left holder between a non-engagement position for the right holder and an engagement position for the right holder, in which an element to be brought into engagement with the user's right eye can be brought into engagement with the user's right eye. This further simplifies handling. For example, the user himself can hold the eye open with one hand and move the respective holder for the eye from the non-engagement position to the engagement position with the other hand.

The holder can be adapted to move back and forth between an adjustable predefined non-engagement position and/or an adjustable predefined engagement position. In other words, the non-engagement position and the engagement position (treatment position) can be predefined. The device with the holder is adapted such that movement is possible, but only up to the predefined engagement position. The predefined engagement position, up to which movement can take place, in particular in the direction of the eye opposite to the direction of gaze, can be adjustable and individually adapted for the user. The engagement position can be adjusted by adjusting the movement of the holder, for example by setting a stop or a limiting device. However, it is also possible that the adjustment of the engagement position is carried out by adjusting one or more elements of the holder, for example by adjusting a length and/or a distance. Thus, even with a predetermined stop, different positions are possible in which the element to be brought into engagement with the eye of the user can be brought into engagement with the eye of the user in the engagement position. Optionally, the non-engagement position can also be predefined. For example, the holder can be adapted to lock or to be detachably locked into a predefined non-engagement position. This facilitates handling in a defined state without causing unintended movement, such as the element to be brought into engagement with the eye of the user hitting the eye of the user too quickly.

The device can be adapted such that, in the predefined engagement position, movement of the holder toward the eye of the user is mechanically limited, in particular wherein the limitation can be adjustable. In other words, in the engagement position (treatment position), further movement beyond the limitation can be mechanically prevented, in particular in the direction of the eye against the direction of gaze. For example, this can prevent the element to be brought into engagement with the eye of the user from exerting excessive pressure on the eye. The limitation can be an adjustable limitation that can be customized for the user to provide a desired predefined engagement position. However, it is also a possibility that the engagement position is set by adjusting one or more elements of the holder, which in the engagement position can rest or abut against a non-adjustable predetermined stop or limiting device.

The device with the holder can be adapted such that the holder in the predefined engagement position abuts against an end stop provided by the frame. In other words, in the engagement position (treatment position), further movement beyond the engagement position can be prevented by the end stop. An end stop can refer to a defined mechanical limit, which can optionally be adjustable. For example, this can prevent the element to be brought into engagement with the eye of the user from exerting too much pressure on the eye. The end stop can be an adjustable end stop, which can be adjusted for the user to provide a desired predefined engagement position.

The holder can be a pivotable holder that is adapted to pivot back and forth between the non-engagement position and the predefined engagement position. An advantage can be that a pivoting movement provides easy handling and enables comfortable placement of the element to be brought into engagement with the eye of the user.

A radius for the pivoting movement can preferably be between 1 cm and 7 cm, in particular between 2 cm and 5 cm, in particular between 2.5 cm and 4 cm. For example, the holder can be provided with a rotary handle, with which the holder can be pivoted back and forth between the non-engagement position and the engagement position. The above-mentioned radii provide an advantageous relationship between the angular velocity of the pivoting movement with which the rotary handle can be operated and the speed at which the element is brought into contact with the eye of the user. If the movement is too slow, there is a risk that the user will blink during engagement. If the movement is too fast, however, there is a risk that engagement will be perceived as uncomfortable.

The device with the holder can be adapted such that the holder can be moved back and forth between the non-engagement position and the engagement position via at least one predefined intermediate position, which is located between the non-engagement position and the engagement position. In particular, a latching device can be provided to hold the holder in the predefined intermediate position. For example, the element to be brought into engagement with the eye of the user can with the predefined intermediate position be brought into proximity of the eye in a first step and only be brought into engagement in a further step. This can further facilitate handling, in particular for visually impaired users.

The holder can be adapted to move the element to be brought into engagement with the eye of the user into the engagement position by a rotary movement, wherein an axis of rotation for the rotary movement is a vertical axis. A vertical axis in this context refers to a vertical axis regarding a wearing position of the device on the head of the user. An advantage of this embodiment can be that the holder can be moved into the engagement position, for example, by swiveling or pivoting it sideways, and minimal leverage effect acts on the eye.

The holder can be adapted to move the element to be brought into engagement with the eye of the user into the engagement position by a rotary movement, wherein an axis of rotation for the rotary movement is a horizontal axis. A horizontal axis in this context refers to a horizontal axis regarding a wearing position of the device on the head of the user. An advantage of this embodiment can be that the element to be brought into engagement with the eye of the user can be brought into engagement with the eye of the user, for example, from the front (opposite to the direction of gaze), in particular at an angle from below from the front. This can facilitate insertion through open eyelids without getting caught on the eyelashes.

In a further refinement, the horizontal axis can lie in a vertical direction above the eye of the users. The holder can be arranged to place the element to be brought into engagement with the eye of the user horizontally on the eye of the user. The holder can be adapted to apply onto or bring into engagement the eye of the user the element to be brought into engagement with the eye of the user by an eccentric swivel movement. This enables easy handling and, at the same time, an advantageous trajectory for the element to be brought into engagement with the eye of the user into the engagement position.

The holder can have an actuating handle for moving the holder between the non-engagement position and the engagement position. In particular, the actuating handle can be arranged in the axial direction along the axis of rotation. An advantage of this embodiment can be ease of use. The actuating handle can be a rotary handle. An advantage of this embodiment may be that an actuating handle in the form of a rotary handle can also be easily actuated with motor impairments of the fingers, as is often the case with elderly people. Hereby, the axis of rotation for rotation into the engagement position can lie within the rotary handle. In particular, the axis of rotation can coincide with a central axis of the rotary handle. An advantage of this embodiment can be that the torque associated with the rotation of the rotary handle is independent of the settings or distances of the individual sections of the electrode holders from the axis of rotation and, in particular, does not depend on the settings of the lengths of the legs for the electrode holders. The actuating handle can in particular be a rotary handle which is arranged in the temporal direction besides the holder. In other words, the rotary handle can be arranged in the axial direction, in particular to the right of a right-hand holder or to the left of a left-hand holder. The arrangement of rotary handles on the left and/or right can in particular allow a one-handed rotation when the device is in place. Arranging the rotary handle in line and in extension with the axis of rotation can, in particular, prevent unwanted force being exerted on the eye. For example, a lever effect, that could occur with an actuating handle not arranged in the axial direction along the axis of rotation, can be avoided or at least reduced.

The holder can comprise an actuating handle for moving the holder between the non-engagement position and the engagement position. The actuation handle can protrude/project (in horizontal direction) beyond the frame of the device. An advantage of this embodiment can be ease of use. In particular, an actuation handle protruding horizontally beyond the frame can be operated without touching and possibly contaminating the element to be brought into engagement with the eye of the user. The actuating handle, in particular a rotary handle, can be arranged temporally at least partially outside the frame. An advantage can be that the actuating handle does not obscure the central field of view when the holder is in the engagement position.

The holder can comprise an actuating handle for moving the holder between the non-engagement position and the engagement position, wherein the device is adapted such that the actuating handle is arranged outside the user's field of view, in particular outside the user's central field of view. In particular, the device can be arranged such that the actuating handle is arranged outside a central field of view of the user in the engagement position. The actuating handle can be a rotary handle. The electrode holder and actuating handle can be adapted and arranged such that, in particular, the user's central field of view remains free, especially in the engagement position and/or optionally in the non-engagement position. An advantage of this embodiment can be that handling is further improved. In particular, with the proposed arrangement outside the field of view, the likelihood of an unwanted eyelid closure reflex when applying a stimulation electrode to the eye of the user can be reduced.

The holder can be adapted to be moved back and forth between the non-engagement position and the predefined engagement position single-handedly and/or without tools. In other words, the device with the holder can be adapted accordingly. By configuring the device with the holder such that the holder can be moved back and forth between a non-engagement position and a predefined engagement position without tools, it is clarified that this is a movement that the user can easily perform multiple times during operation. In particular, the movement can be performed by the user themselves after the device has already been adjusted to the user, in particular after the device has already been placed on the head of the user. Preferably, no friction connection is provided. By adapting the device with the holder such that the holder can be moved back and forth between the non-engagement position and the predefined engagement position with one hand or single-handedly, the user has another hand free, for example, to keep the eye open with the other hand. This further improves handling, in particular for a visually impaired user who wants to use the device without assistance.

The device can be adapted such that adjustments to all variable parts and their locking in the selected settings can be performed without tools. An advantage can be that adjustments can be made in a simple way.

The holder can be adapted such that the element to be brought into engagement with the eye of the user can be brought into the predefined engagement position from the front of the eye of the user at an angle in the range of +/−30°, in particular at an angle in the range of +/−20° to the horizontal. An advantage of this embodiment can be that insertion through the eyelids is simplified. In particular, a predefined pivoting movement can take place, wherein, when inserting, preferably no “twisting” or sawing movement of the element occurs on the eye.

The holder can comprise a horizontal crossbar which is arranged at the frame adjusted in the horizontal direction.

An operating element for moving the holder in a horizontal direction relative to the frame can be arranged in the actuating handle. In other words, the actuating handle for moving the holder between the non-engagement position and the engagement position can be arranged together, wherein the operating element is arranged in the actuating handle. This structure can enable a coaxial arrangement of the actuating handle and the operating element. For example, an axis for a horizontal pivoting movement can coincide with an axis in whose longitudinal direction the holder is adjusted in the horizontal direction relative to the frame. This can enable a compact structure. In addition, this arrangement with the same axes can allow an intuitive operation. It can thus be sufficient to provide only one common arrangement comprising operating element and actuating handle per electrode holder. The proposed arrangement can facilitate operation, in particular for visually impaired users. The commonly arranged operating elements could be easily detected tactile and actuated.

The holder can comprise a nasal vertical leg (vertical arm) and a temporal vertical leg (vertical arm), which extend downward in a vertical direction from the holder, in particular from a horizontal crossbar of the holder. The nasal vertical leg and/or the temporal vertical leg can optionally be length adjustable. The length adjustment of one or both of the legs can thus enable height adjustment of the element to be brought into engagement with an eye of the user with respect to the eye of the user.

The holder can comprise a nasal horizontal leg (horizontal arm) and a temporal horizontal leg (horizontal arm), wherein the nasal horizontal leg comprises a first receptacle for the element to be brought into engagement with the eye of the user, wherein the temporal horizontal leg comprises a second receptacle for the element to be brought into engagement with the eye of the user. The nasal horizontal leg and/or the temporal horizontal leg can optionally be length adjustable. The length adjustment of one or both of the legs can thus enable the distance adjustment between the element to be brought into engagement with the eye of the user with respect to the eye of the user. Thanks to the length adjustment(s), it is thus possible to easily adjust where the element to be brought into engagement with the eye of the user comes to rest when the holder is in the predefined engagement position.

The first receptacle of the nasal horizontal leg and the second receptacle of the temporal horizontal leg can have a fixed horizontal distance between them. An advantage of this specific embodiment can be that an element to be received by the two receptacles does not have to be adjusted for different distances.

For example, the element to be brought into engagement with an eye of the user can be a stimulation electrode arrangement. The stimulation electrode arrangement can comprise a first electrode carrier and a second electrode carrier, wherein the stimulation electrode is arranged between the first electrode carrier and the second electrode carrier. The stimulation electrode arrangement can be provided in a blister pack in which the first and second electrode carriers are arranged at a defined distance from each other, wherein the defined distance corresponds to the fixed horizontal distance between the first receptacle of the nasal horizontal leg and the second receptacle of the temporal horizontal leg. Even though the holder is adjustable, the fixed distance between the receptacles allows easy removal directly from a predetermined arrangement in which the elements to be received are also provided at the fixed predetermined distance. An effort required for inventory of stimulation electrode arrangements can thus be reduced. Furthermore, hygiene can be improved, as the stimulation electrode does not have to be removed by hand and the distance between the two electrode carriers does not have to be adjusted manually.

In an advantageous embodiment, the nasal vertical leg and the temporal vertical leg can extend downward from the horizontal crossbar of the holder in a vertical direction. The nasal horizontal leg can be arranged at a lower end of the nasal vertical leg. The temporal horizontal leg can be arranged at a lower end of the temporal vertical leg. In particular, the components can be arranged such that a free optical path is provided in the user's gaze direction.

In a further refinement, the nasal vertical leg and the temporal vertical leg can be arranged orthogonal to the crossbar of the holder. The nasal horizontal leg and the temporal horizontal leg can be arranged orthogonal to the crossbar of the holder. The nasal horizontal leg can be arranged orthogonal to the nasal vertical leg. The temporal horizontal leg can be arranged orthogonal to the temporal vertical leg. An advantage of this arrangement can be that the device is flexible and adaptable to the user in a simple way with elements in a defined orientation with respect to each other. Furthermore, even when the holder is in the predefined engagement position, a free optical path can be provided for the user. This can improve the perceived wearing comfort during treatment, for example during electrostimulation of the eye.

The nasal vertical leg and/or the temporal vertical leg and/or the nasal horizontal leg and/or the temporal horizontal leg can each be length adjustable respectively with a threaded spindle. For example, a respective rotary handle can be provided for actuation of the threaded spindle. During the present application an accurate adjustment of the desired length is thus possible.

The holder can be adapted to bring the element to be brought into engagement with the eye of the user at a first support point and a second support point into engagement with the eye of the user in the engagement position. The holder can be adapted to adjust the first support point in three spatial directions and to adjust the second support point in three spatial directions. However, preferably exactly one spatial direction is coupled such that the first and second support points can have a fixed distance in this spatial direction. An advantage of this solution may be that reliably reproducible support points can be provided. Two support points can be provided per eye, each of which can be adapted to the user in three coordinate axes. A distance or separation can be advantageously fixedly defined, such that, for example, an element to be brought into engagement with the eye of the user with a defined separation can be received, for example from a package, in particular a blister pack, as exemplarily described herein.

Various elements to be brought into engagement with the eye of the user are conceivable. Within the scope of the present disclosure, an element to be brought into engagement with an eye of the user can also refer to an element to be applied on an eye of the user. As mentioned at the outset, the element to be brought into engagement with the eye of the user can be a stimulation electrode or stimulation electrode arrangement. The element to be brought into engagement with the eye of the user can also be a contact lens; wherein the holder is a holder for the contact lens and is adapted to apply the contact lens to the eye of the user in the predefined engagement position.

Furthermore, aspects of the present disclosure relate to a system for electrostimulation of the eye with a device (as disclosed herein) and a stimulation electrode arrangement for electrostimulation of the eye, wherein the element to be brought into engagement with an eye of the user is a stimulation electrode and wherein the holder is an electrode holder for the stimulation electrode. The holder and the device can be adapted as described above or as exemplarily explained in the present disclosure.

The holder can hold the stimulation electrode directly or indirectly. The stimulation electrode arrangement comprises the stimulation electrode. However, the stimulation electrode arrangement can also comprise, for example, one or more electrode carriers and be attached to the holder via the electrode carriers.

Here, the nose part can have a nose clip and two eye brackets, to which respectively one electrode holder is attached.

Here, the eye brackets furthermore are preferably electrically insulated from one another and contacted individually such that a therapy with different current strengths for the two eyes is possible.

Here, the arrangement for holding the frame has two earpieces which are fixed to the nosepiece, preferably fixed thereto in foldable fashion, in a manner known per se. The arrangement for holding the frame can also comprise side arms of variable width, in particular spring-loaded variable width side arms.

The frame can have a forehead abutment (forehead stop). When placed on the head, the forehead abutment can rest against the user's forehead and limit movement of the device in the direction towards the eyes of the user. This can be advantageous compared with a device that only has a nose part and which can be moved back and forth on the back on the nose ridge. In particular, the forehead abutment can further improve positioning accuracy also in the engagement position and preferably prevent excessive pressure from being exerted on the eye of the user by the element to be brought into engagement with the eye of the user in the engagement position.

In general, it is further preferable for a device and system for electrostimulation, if a counter electrode for the stimulation electrode is provided.

Furthermore, aspects of the present disclosure relate to a method, in particular a method for applying a stimulation electrode of a device for electrostimulation of the eye to the eye of the user, wherein the method comprises the following steps:
a) providing a device, in particular as described in the present disclosure;
b) placing (putting on) the device on the head of the user, wherein the holder is in the non-engagement position; and
c) moving the holder from the non-engagement position to the predefined engagement position after placing the device on the head of the user to bring the element to be brought into engagement with the eye of the user into engagement with the eye of the user.

The optional further embodiments and refinements described for the device according to the first aspect apply accordingly to the further aspects of the present disclosure, i.e., in particular to the system, in particular for electrostimulation of the eye, and the method.

Further advantages are apparent from the description of the accompanying drawing.

It is to be understood that the features mentioned above and those to be explained below can be used not only in the respective combination specified, but also in other combinations or in isolation, without departing from the scope of the present disclosure.

### BRIEF DESCRIPTION OF THE DRAWINGS

Embodiments of aspects of the disclosure are illustrated in the drawings and are explained in more detail in the following description.

FIG. 1 is a schematic perspective view of a device for electrostimulation of an eye, according to the present disclosure, with a holder in a non-engagement position;

FIG. 2 is a schematic side view of a device for electrostimulation of an eye, according to the present disclosure, with a holder in a non-engagement position;

FIG. 3 is a schematic side view of a device for electrostimulation of an eye, according to the present disclosure, with a holder in a predefined engagement position;

FIG. 4 is a schematic perspective illustration of a spectacle-like frame, according to the present disclosure;

FIG. 5 is a schematic side view of a holder, according to the present disclosure, illustrated in a first state;

FIG. 6 is a schematic side view of a holder, according to the present disclosure, illustrated in a second state;

FIG. 7 is a schematic perspective illustration of a holder, according to the present disclosure, with an electrode arrangement for electrostimulation of an eye;

FIG. 8 illustrates a flow chart of a method for attaching a stimulation electrode arrangement to a device for electrostimulation of an eye, according to the present disclosure;

FIG. 9 is a schematic view of a section of a device for electrostimulation of an eye, according to the present disclosure, with a holder with an electrode arrangement;

FIG. 10 is a schematic view of an actuating handle with a corresponding latching device, according to the present disclosure;

FIG. 11 is a schematic perspective view of a section of a device for electrostimulation of an eye, according to the present disclosure, with a holder with an electrode arrangement;

FIG. 12 is a schematic cross-sectional view of the actuating handle and the corresponding latching device of FIG. 10; and

FIG. 13 is a schematic cross-sectional view of a section of a device for electrostimulation of an eye, according to the present disclosure.

### DETAILED DESCRIPTION

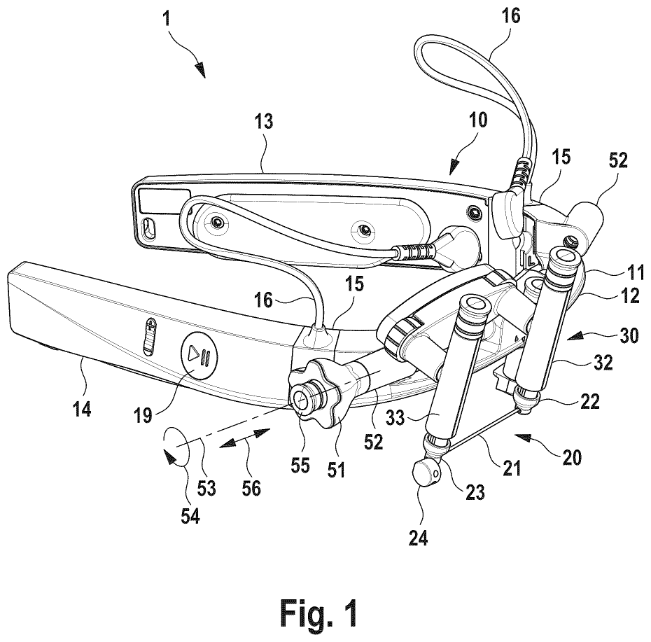

FIG. 1 shows, as an example for illustration purposes, a system 1 for electrostimulation of the eye of a user or patient. The system 1 comprises a device 10 with a holder 30 and an element 20 to be brought into engagement with the eye of the user. In the present example, a device for electrostimulation of the eye is provided, in which a stimulation electrode arrangement 20 is to be brought into engagement with the eye.

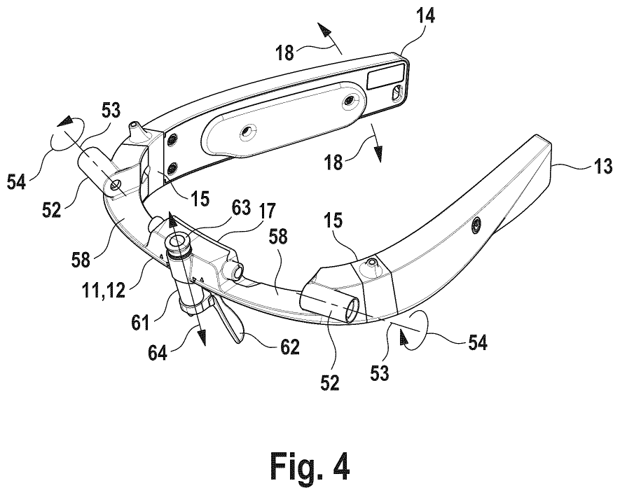

The device 10 comprises a spectacle-like frame 11 with a nose part 12 and an arrangement connected to the nose part 12 for holding the frame on the head of the patient. The arrangement for holding the frame 11 on the head of the patient can comprise two temples or side arms 13, 14, as is usual with spectacles. Optionally, the side arms 13, 14 can be attached to the nose part 12 in a foldable manner. The side arms 13, 14 can also be connected to the nose part 12 in a width-variable way via spring elements 15. In this way, it is possible to flexibly adapt the device to the user's head, as shown by the arrows 18 in FIG. 4.

The device or frame 11 can optionally comprise a forehead abutment 17, as shown in FIG. 4. In a worn state, the forehead abutment can rest or abut against the user's forehead and limit movement of the device in a direction toward the eyes of the user. This can be advantageous compared with a device without a forehead abutment, which, for example, only has a nose part and which can be moved back and forth on the nose ridge. For example, this can prevent the element to be brought into engagement with the eye of the user from exerting too much pressure on the eye.

The device also comprises a holder 30 arranged on frame 11 for an element to be brought into engagement with the eye of the user. The holder 30 is adapted to be moved back and forth between a non-engagement position and a predefined engagement position.

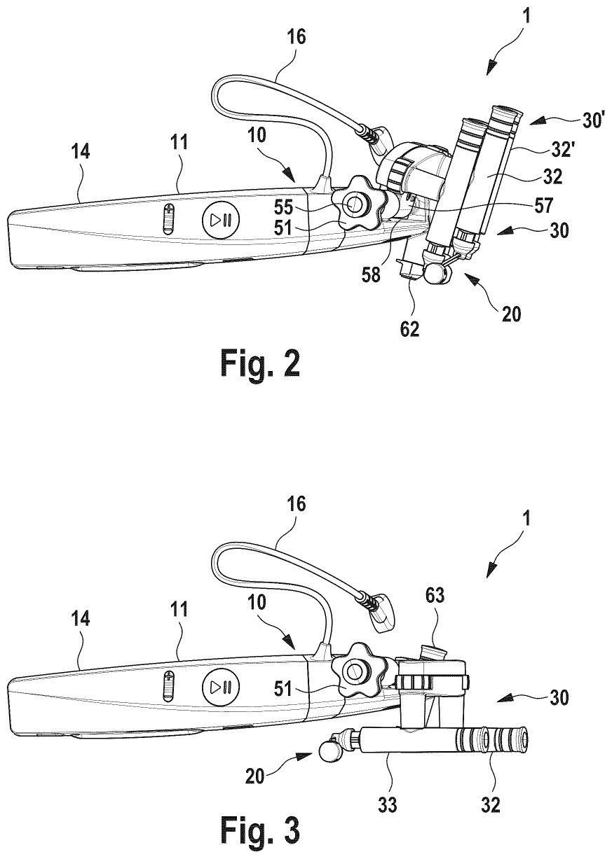

FIGS. 1 and 2 show the holder 20 in the non-engagement position. FIG. 3 shows the holder 20 in the predefined engagement position. FIG. 4 illustrates the frame 11 without the holder.

In the present example, the holder 30 is an electrode holder for a stimulation electrode arrangement 20 with a stimulation electrode 21. The holder 30 can be arranged to pivot back and forth between the non-engagement position and the predefined engagement position. In particular, the pivotable holder 30 can be moved back and forth between a raised position, as the non-engagement position, and a defined treatment position, as an engagement position. In the example shown in FIG. 1, the holder is in a raised position. This enables a patient to place the spectacle-like frame 11 on their head with the holder 30 in the non-engagement position. This makes handling easier. In a first step, only the spectacle-like frame is put on; it is however not yet necessary to apply the stimulation electrode 21 onto the eye in the same step.

Only after the spectacle-like frame 11 has been placed on the head is the holder 30 lowered and moved into the predefined engagement position. In the case of a device for electrostimulation of the eye, the stimulation electrode 21 can come into contact or engagement with the front surface of the eye in the engagement position. The stimulation electrode 21 preferably curves outwards so that it can fit snugly against the curvature of the eye surface, in particular the cornea. This is possible because the stimulation electrode 21 is flexible and can be held flexibly in the second electrode carrier 23 via the weight body 24.

The device 10 preferably comprises two separate holders 30, 30′. For simplicity, only one holder is illustrated in FIGS. 1 and 3. The second holder 30′ is schematically illustrated in FIG. 2 behind the first holder 30. The device 30 can comprise a left holder 30′ for an element to be brought into engagement with a left eye of the user, in particular for a stimulation electrode 21 for the left eye, and a right holder 30 for an element to be brought into engagement with a right eye of the user, in particular for a stimulation electrode 21 for the right eye. The left holder 30′ and the right holder 30 are adapted to be moved separately from each other between a respective non-engagement position and a respective engagement position. The features described for the right holder 30 apply accordingly to the left holder 30′.

The arrangement of the separate holders for the right and left eyes can also be seen in the perspective view of the spectacle-like frame 11 in FIG. 4, with the respective receptacles 52 in which the rotary axes for the separate holders for the right and left eyes can be mounted.

In the example shown in FIG. 1, the holder 30 can, for example, be moved back and forth between the raised non-engagement position (see FIG. 2) and the defined engagement position (see FIG. 3) via an actuating handle 51 arranged at the side. The actuating handle 51 projects or protrudes horizontally beyond the frame 11.

The holder 30 is hereby mounted on the spectacle-like frame 11 such that it can by the receptacles 52 be rotated or pivoted. For example, with a bearing in the receptacle 52, as illustrated for an electrode holder which is not shown for the left eye. The axis of rotation is designated with 53. The rotation is designated with reference sign 54. In the present example, the horizontal axis 53 lies in the vertical direction above the eyes of the user and therefore does not interfere with the user's gaze. Alternatively, the axis of rotation for the rotational movement can be a vertical axis.

In the embodiment shown in FIG. 1, the horizontal axis of rotation 53 in the engagement position is also further away from the eye of the user in the direction of view than the stimulation electrode 21. As a result, the swiveling movement into the eye is essentially horizontal, but with a slight angle from below, in order to bring the stimulation electrode 21 into engagement with the eye of the user. This has proven to be advantageous, as the lower lid can be pulled down slightly by the user during swiveling. The eyelashes are thus not in the way and engagement is facilitated.

However, other configurations are also possible. Preferably, the holder is adapted to bring the element to be brought into engagement with the eye of the user into the predefined engagement position from the front of the eye of the user at an angle in the range of +/−30°, in particular at an angle in the range of +/−20° to the horizontal.

Separate holders 30, 30′ can be provided for the right eye and the left eye. For simplicity, only one holder 30, here an electrode holder, is illustrated in FIG. 1.

The stimulation electrode arrangement 20 is attached to the electrode holder 30, preferably magnetically. In the shown example, the stimulation electrode arrangement 20 comprises a first electrode carrier 22 and a second electrode carrier 23. The stimulation electrode 21 is arranged between the first electrode carrier 22 and the second electrode carrier 23. The first and/or second electrode carrier are adapted to be magnetically attached to the electrode holder 30.

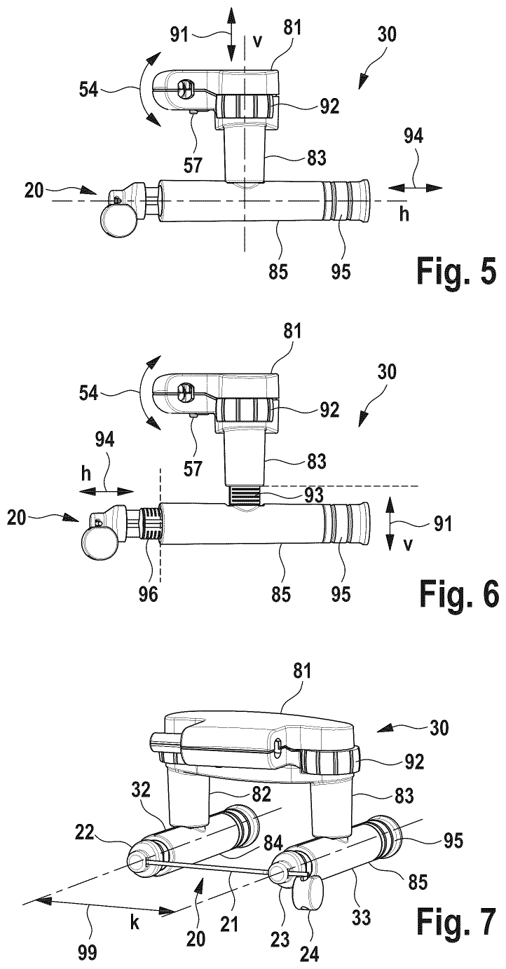

In the example shown in FIG. 1, the electrode holder 30 can comprise a first, nasal electrode holder part 32 and a second, temporal electrode holder part 33. The first, nasal electrode holder part 32 can also be referred to as the nose-side electrode holder part. The second, temporal electrode holder part 33 can also be referred to as the temple-side electrode holder part. The first electrode carrier 22 is a nasal electrode carrier and is adapted to be magnetically attached to a receptacle of the nasal electrode holder part 32. The second electrode carrier 23 is a temporal electrode carrier and is adapted to be magnetically attached to a receptacle of the temporal electrode holder part 33. The configuration of an exemplary holder 30 is explained in more detail further below with reference to FIGS. 5 to 8.

In an assembled state, the stimulation electrode 21 is electrically conductively connected to a stimulation device for electrostimulation of the eye. Preferably, the electrical connection is provided via the electrode holder 30. The stimulation device generates the desired electrical signals for electrostimulation. Any stimulation device can be used as the stimulation device that can emit suitable stimulation signals, in particular sine waves or pulsed electrostimulation signals for the treatment of the eye.

The stimulation device can be integrated into the spectacle-like frame 11. This is schematically illustrated in FIG. 1 by reference sign 19 and control elements for starting or pausing the electrostimulation. However, the stimulation device can also be provided as an external stimulation device.

In order to close the electric circuit for electrostimulation, counter electrodes 16 can also be provided, which can, for example, be attached to the patient's forehead or earlobe.

In the context of the present disclosure, the stimulation device may be a device for stimulating and/or measuring. For example, a stimulation signal can be generated which can be applied via the stimulation electrode arrangement 20 with the stimulation electrode 21. However, it can also, for example, be an ERG apparatus with which an electroretinogram can be recorded, which is acquired with the stimulation electrode 21 of the stimulation electrode arrangement 20.

One or more predefined intermediate positions (not illustrated) can be provided between the non-engagement position shown in FIG. 2 and the predefined engagement position shown in FIG. 3. The holder 30 can be adapted to be moved back and forth between the non-engagement position and the engagement position via at least one predefined intermediate position located between the non-engagement position and the engagement position, in particular wherein a latching device is provided to hold the holder 30 in the predefined intermediate position.

To adjust the positioning of the holder 30 relative to the frame 11, the holder 30 can be adapted to be moved in a horizontal direction 56, i.e., in the longitudinal direction of the axis 53. This enables an adjustment of the device 10 for different eye distances. An operating element 55 for this horizontal displacement, for example by a threaded spindle arranged in the holder 30, can be arranged in the actuating handle 51, as exemplarily shown in FIGS. 1 and 2.

As shown in FIGS. 2 and 4, an end stop 58 can be provided at the frame 11, against which the holder 30 abuts in the predefined engagement position. For this, the holder 30 can comprise a corresponding abutment surface 57, as shown in FIG. 5. In the predefined engagement position, the abutment surface 57 of the holder 30 rests against the end stop 58 of the frame 11, as shown in FIG. 3. Accordingly, the abutment surface 57 does not abut against the end stop 58 in the non-engagement position.

Alternatively, the predefined engagement position can also be limited or defined in other ways, for example by limiting the rotation 54 about the axis of rotation 53. This can also be achieved, for example, with an end stop in the respective receptacles 52.

The side view in FIG. 2 also shows a nose support 62 with which the frame 11 of the device 10 can be supported on the user's nose. The nose support can also be adjustable. For example, the nose support 62 can be adjustable in length or height 64 with a control element 63, as shown in FIGS. 3 and 4.

With reference to FIGS. 5-7, an embodiment of a holder according to aspects of the present disclosure is explained in more detail. The holder 30 is adapted to be arranged at a frame 11, as shown, for example, in FIGS. 1-4, wherein the device 10 with the frame 11 and the holder 30 is adapted to be moved back and forth between a non-engagement position and a predefined engagement position. For example, by a pivoting movement as schematically illustrated by arrow 54. In order for the element to be brought into engagement with the eye of the user to be correctly positioned, the holder 30 can provide several adjustment options.

The example shown again relates to a device for electrostimulation of the eye with a stimulation electrode arrangement 20 comprising a nasal electrode carrier 22, a temporal electrode carrier 34, and a stimulation electrode 21 arranged between them, to which a weight body 24 is connected in order to tension the stimulation electrode 21. The nasal electrode carrier 22 is attached to an adjustable nasal electrode holder part 32. The temporal electrode carrier 23 is attached to an adjustable temporal electrode holder part 33. A distance 99 in the horizontal direction is preferably a constant distance k. The stimulation electrode arrangement can also be provided, for example in a blister package, such that the nasal electrode carrier 22 and the temporal electrode carrier 23 are provided with the same constant distance k and can be attached in a simple manner.

As shown in FIGS. 5 and 6, the holder 30 can comprise a stop surface 57, in particular an adjustable stop surface 57. Depending on how far the stop surface 57 protrudes, the inclination of the holder 30 relative to the frame 11 can be varied with the end stop 58 (see FIG. 4).

As shown in FIGS. 5-7, the holder 30 comprises a horizontal crossbar 81. The holder also comprises a nasal vertical leg 82 and a temporal vertical leg 83, which extend downward in a vertical direction from the horizontal crossbar 81 of the holder 30. The nasal vertical leg 82 and the temporal vertical leg 83 are arranged orthogonally to the crossbar 81 of the holder 30. In the context of the present disclosure, “orthogonal” refers to a substantially orthogonal arrangement, in particular an arrangement at an angle of 90°+/−30°, in particular +/−20°, in particular +/−10°. The nasal vertical leg 82 and/or the temporal vertical leg 83, preferably both, are adjustable in length. This allows the position of the element to be brought into engagement with an eye of the user, in this case the stimulation electrode arrangement 20, to be adjusted. This is shown in FIG. 6 by arrow 91 for an adjustment in the vertical direction v.

In the side view of FIG. 5, the temporal vertical leg 83 is shown in the retracted state. In the side view in FIG. 6, the temporal vertical leg 83 is shown in the extended state 93. For example, the adjustment can be made by a rotary wheel 92 and an (internal) threaded spindle. Several markings can be provided to facilitate a defined length adjustment. An advantage of the proposed solution can be that a tool-free adjustment is enabled. In particular, the proposed solution with threaded spindles allows for fine adjustment and, at the same time, easy operation for visually impaired users. The lengths of the legs 83 can, for example, be changed without tools by threaded spindles and optionally fixed after manual adjustment.

As shown in FIGS. 5-7, the holder 30 can further comprise a nasal horizontal leg 84 and a temporal horizontal leg 85. The nasal horizontal leg 84 is arranged at a lower (length-adjustable) end of the nasal vertical leg 82. The temporal horizontal leg 85 is arranged at a lower (length-adjustable) end of the temporal vertical leg 83. The vertical and horizontal legs can be arranged orthogonally to each other. The nasal horizontal leg 84 comprises a first receptacle for the element to be brought into engagement with the eye of the user, here for the first electrode carrier 22 of the stimulation electrode arrangement 20. The temporal horizontal leg 85 comprises a second receptacle for the element to be brought into engagement with the eye of the user, here for the second electrode carrier 23 of the stimulation electrode arrangement 20. The nasal horizontal leg 84 and/or the temporal horizontal leg 85, preferably both, are adjustable in length. Thereby the position of the element to be brought into engagement with the eye of the user, here the stimulation electrode arrangement 20, can be adjusted. This is illustrated in FIG. 6 by the arrow 94 for adjustment in the horizontal direction h.

The side view in FIG. 5 shows the temporal horizontal leg 85 in the retracted position. The side view in FIG. 6 shows the temporal horizontal leg 85 in the extended position 96. For example, adjustment can be made by a rotary wheel 95 and an (internal) threaded spindle. Several markings can be provided to facilitate a defined length adjustment.

As shown in FIG. 7, a free optical path can be provided between the legs 82, 83 of the holder in the direction of gaze of the user. This increases wearing comfort and user acceptance, in particular during longer applications.

Once the electrode holder 30 has been adjusted, the electrode holder can with the fixed adjusted configuration be moved back and forth between the non-engagement position and the defined engagement position.

It is to be understood that the holder can also be adapted for another element to be brought into engagement with the eye of the user, such as a contact lens. In this case, it can be sufficient to provide only one vertical leg 83 and horizontal leg 85 arranged centrally on the crossbar 81 with a receptacle for example for a contact lens.

The holder itself does not necessarily have to directly contact the eye of the user. One or more intermediate elements can also be provided, as shown in the present example by the stimulation electrode arrangement. Furthermore, engaging with the eye of the user is not limited to contacting the cornea, but also by contacting one or more regions, in particular skin areas, in the region of the eye. Transcutaneous measurement and/or stimulation is also conceivable.

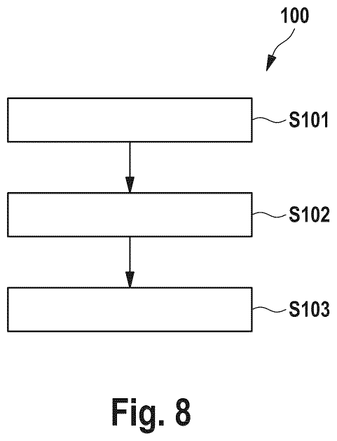

FIG. 8 shows a flowchart of a method 100, in particular a method for applying a stimulation electrode of a device for electrostimulation of the eye to the eye of the user.

In a first step S101, a device as disclosed herein is provided.

In a second step S102, the device is placed on the head of the user, wherein the holder is in the non-engagement position.

In a third step S103, after the device has been placed on the head of the user, the holder is moved from the non-engagement position to the predefined engagement position to bring the element to be brought into engagement with the eye of the user into engagement with the eye of the user.

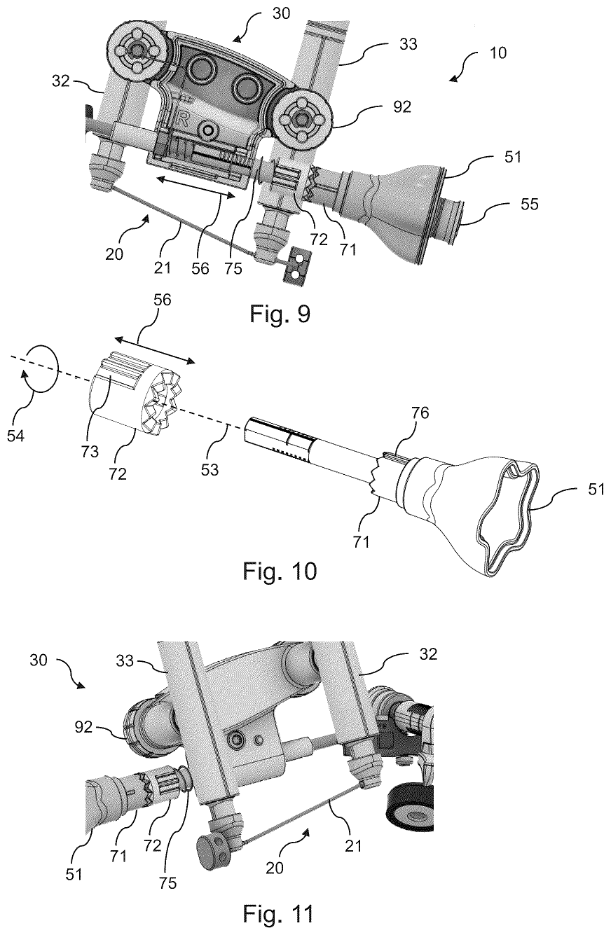

Further possible embodiments of the proposed device 10 are explained in the following with reference to FIGS. 9-13. It shall be understood that these are merely exemplary embodiments and that the present disclosure is not limited thereto. Elements that have already been introduced above are designated by the same reference signs. In the following, optional advantageous further refinements and/or differences will be highlighted.

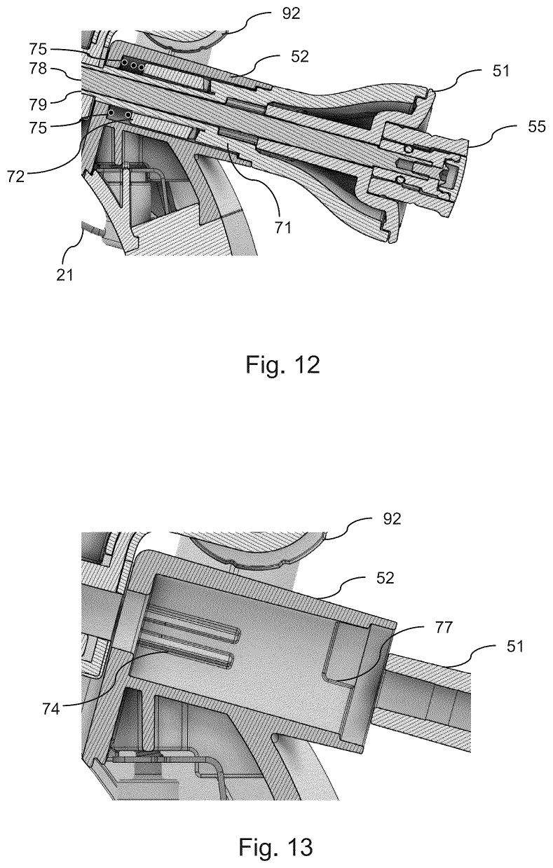

FIG. 9 shows a schematic partial sectional view of a section of components of the device 10 with the holder 30 with an electrode arrangement 20 in a view from a top side. Here, a sectional view of the holder 30 is shown. The holder 30 is mounted by the receptacles 52 on the spectacle-like frame 11 such that it can be rotated or pivoted (see FIG. 4). However, the receptacle 52 is not shown in FIGS. 9 and 10 in order to illustrate the internal elements. The receptacle 52 is shown in the illustrations in FIGS. 12 and 13.

In the example shown, a latching device is provided in receptacle 52. The latching device can be adapted to hold the holder 30 in the predefined engagement position but also in a non-engagement position. As already described above, one or more predefined intermediate positions can be provided between the non-engagement position shown in FIG. 2 and the predefined engagement position shown in FIG. 3. The holder 30 can be adapted to move back and forth between the non-engagement position and the engagement position via at least one predefined intermediate position located between the non-engagement position and the predefined engagement position. In the present example, the latching device is adapted to hold the holder 30 in several predefined intermediate positions. The latching device can be adapted to provide a respective releasable form-fit connection in different latching positions in order to hold the electrode holder 30 relative to the frame 11 in the predefined engagement position, in the non-engagement position, and optionally in one or more intermediate positions.

For example, the latching device can be provided by corresponding latching elements 71, 72. A first latching element 71 is arranged on the holder 30 or on the actuating handle. A second latching element 72 is arranged at the frame 11, in particular on the receptacle 52. The latching elements can, for example, be configured as corresponding gear rims which can engage with each other in several defined angular positions. The latching elements are thus configured to provide defined form-fit connections in various (predetermined) angular positions. In an embodiment, a first and a second angular position differ by at least 5°, in particular by at least 15°, in particular by at least 30°, in particular by at least 45°, in particular by at least 60°. Alternatively or in addition, a first and a second angular position differ by at most 270°, in particular by at most 225°, in particular by at most 180°, in particular by at most 150°. The difference refers to a maximum angle between the defined engagement position and a non-engagement position. Thus, a limited angle range may be provided for a pivoting movement, which can facilitate handling.

In the context of the present disclosure, preferably no frictional lock connection is provided. In the case of a friction lock connection, a force counteracting the frictional force must be applied in order to release the friction lock connection. This force causes a torque with an axis of rotation which, in the case of a movement away from the eye, lies at the point of contact of the side arm on the ear and, in the case of a movement towards the eye, lies at the point of contact of the nose support on the bridge of the nose. If the movement is away from the eye, this causes the entire device to move upwardly, at least until the friction lock is released. To prevent the device mounted on the nose from twisting and slipping, in particular if one of the electrodes has already been brought into engagement with an eye, a counterforce opposing the direction of rotation would have to be exerted with the second hand. This means that both hands must always be on the device in order to pivot the electrodes toward the eye. This would have the disadvantage that neither hand is free to push the eyelids down or upwardly, i.e., to keep the eye open when applying the stimulation electrode.

The proposed form-fit connection by a latching device, on the other hand, can offer one or more advantages. The latching device can be adapted such that a form-fit connection is released by a force acting predominantly parallel to the axis 53 in the bearing 52. This can for example be achieved by two corresponding latching elements 71, 72 arranged in particular on the axis 53. A schematic view of an actuating handle 51 with a first latching element 71 and a corresponding latching element 72 is illustrated in FIG. 10.

By releasing the form-fit latching, no or, at most, a lower force is caused, compared with a friction lock connection, that leads to rotation about an axis of rotation at the point of contact of the side wing or the nose support on the nose ridge. For moving the electrode holder from one latching position to the next, once the form-fit latching has been released, no or, at most, lower frictional forces need to be overcome. This reduces or eliminates the risk that by the rotational movement a stimulation electrode 21 already positioned on the surface of the eye shifts.

A further advantage can be that the rotational movement can be performed with only one hand (single-handed). The second hand remains free, for example, to pull down the lower eyelid so that the stimulation electrode 21 of the stimulation electrode arrangement 20 can be positioned on the lower half of the eye surface without for example during pivoting in touching the eyelashes.

The latching element 72 in the shown embodiment is preferably rotationally fixedly connected to the frame or to the receptacle 52. However, the latching element can be mounted so that it can be displaced in the axial direction along the axis of rotation 54. For this purpose, the latching element 72 and the receptacle 52 can comprise corresponding guide elements 73, 74. In the present example, recesses are provided on the latching element 72 as guide elements 73, as illustrated in FIGS. 9-11. Corresponding webs are provided on the receptacle 52 as guide elements 74, as illustrated in FIG. 13. A spring element 75 can be provided in the receptacle 52, which presses the latching element 72 in the direction of the corresponding latching element 71. The spring element 75 defines a force that must be overcome in the axial direction 56 in order to release the form-fit connection.

In other words, the latching positions in the present embodiment are defined by the interlocking teeth of the first latching element 71 and the second latching element 72. The actuation of the pivoting mechanism via the actuating handle 51 causes the teeth to move against each other. The latching element 72 can be moved in the axial direction. The latching element 72 is fixed by the two elongated grooves or recesses as a guide element 74 in the outer part, i.e., in the receptacle 52, and cannot be rotated. The release to rotate the corresponding latching element 71 is achieved by a spring element 75. Thereby, the flexibility is provided to rotate the latching element 72 and to turn one tooth further. Preferably, the guide element can be provided both on a top side, as shown in the top view in FIG. 9, and on a bottom side, as shown in the view in FIG. 11.

Referring to the illustrations in FIGS. 10 and 12, the holder 30 and the frame 11 can be adapted to mechanically limit a movement relative to each other. In particular, a limit can be set for an angular range of movement relative to each other. The holder 30 can be a pivoting holder. The angular range can be limited to an angular range of not more than 270°, in particular not more than 180°, in particular not more than 150°. In the embodiment shown in FIGS. 10 and 12, the holder 30 and the actuating handle 51 can comprise a stop 76. The receptacle 52 can provide a corresponding stop 77. For example, the stop 76 can be provided by a web or a raised section and the stop 77 can be provided by a corresponding recess or groove with a side surface serving as a stop. A desired angle of rotation can thus be defined in an easy way. It is to be understood that the stops 76, 77 can also be provided multiple times, for example, upwardly and downwardly or offset by 180°. The limitation described here can in the alternative or in addition be provided to the limitation by the stop surfaces 57, 58, as illustrated in FIGS. 2 and 4, in order to provide a predefined engagement position. Alternatively, the predefined engagement position can also be limited or defined in other ways, for example by limiting the rotation 54 about the axis of rotation 53. This can also be achieved, for example, with an end stop in the respective receptacles 52.

FIG. 12 shows a schematic cross-sectional view with the actuating handle 51 and the latching device with the latching elements 71, 72. Optionally, the holder 30 can be pivoted not only relative to the frame 11. In order to adjust the position of the holder 30 relative to the frame 11, the holder 30 can be adapted to be moved in the horizontal direction 56, i.e., in the longitudinal direction of the axis 53. This enables the device 10 to be adjusted for different eye separations. The adjustment in the horizontal direction is illustrated in FIGS. 1 and 9 with arrow 56. An operating element 55 for this horizontal displacement, for example by a threaded spindle arranged in the holder 30, can be arranged in the actuating handle 51, as illustrated in FIGS. 1, 2, 9, and 12. This can be implemented by providing two coaxial shafts 78, 79. For example, an inner shaft 78 can be surrounded by an outer shaft 79. The inner shaft 78 can be coupled with operating element 78. A displacement can be provided, for example, by a threaded rod and a rotary movement on the operating element 55. A rotation of the holder 30 relative to the frame can be provided by a rotary movement on the actuating handle 51.

In conclusion, according to aspects of the present disclosure, the handling of a device, in particular a device for electrostimulation of the eye, can be further improved and a more comfortable user experience can be enabled. In particular, application of an element to be brought into engagement with the eye of the user can be facilitated.

It is to be understood that the foregoing description is of one or more embodiments of the present disclosure. The present disclosure is not limited to the particular embodiment(s) disclosed herein, but rather is defined solely by the claims below. Furthermore, the statements contained in the foregoing description relate to the disclosed embodiment(s) and are not to be construed as limitations on the scope of the present disclosure or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all of the following: “A”; “B”; “C”; “A and B”; “A and C”; “B and C”; and “A, B, and C.”

1-36. (canceled)

37. A device for electrostimulation of an eye, comprising:
a spectacle-like frame with a nose part;
an arrangement connected to the nose part for holding the spectacle-like frame on a head of a user; and
at least one holder arranged at the spectacle-like frame for an element to be brought into engagement with said eye of the user, wherein the element is a wire-shaped stimulation electrode, and wherein the at least one holder is adapted for back and forth movement between a non-engagement position and a predefined engagement position.

38. The device of claim 37, wherein the at least one holder is an electrode holder for a stimulation electrode arrangement.

39. The device of claim 38, wherein the stimulation electrode arrangement comprises a first electrode carrier and a second electrode carrier, wherein the wire-shaped stimulation electrode is arranged between the first electrode carrier and the second electrode carrier, and wherein at least one of the first electrode carrier and the second electrode carrier is adapted to be magnetically attached to the electrode holder.

40. The device of claim 37, wherein the at least one holder comprises two separate holders, the two separate holders including a left holder for an element to be brought into engagement with a left eye of the user and a right holder for an element to be brought into engagement with a right eye of the user, wherein the left holder and the right holder are adapted to be moved separately from each other back and forth between the non-engagement position and the predefined engagement position, respectively.

41. The device of claim 37, wherein the at least one holder is adapted for back and forth movement between an adjustable predefined non-engagement position and an adjustable predefined engagement position.

42. The device of claim 37, wherein in the predefined engagement position, movement of the at least one holder toward said eye of the user is mechanically limited, and wherein the at least one holder in the predefined engagement position abuts against an end stop provided by the spectacle-like frame.

43. The device of claim 37, wherein the at least one holder is a pivotable holder which is adapted to pivot back and forth between the non-engagement position and the predefined engagement position, the at least one holder abutting an end stop in the predefined engagement position, which is adapted to limit a pivoting movement between the non-engagement position and the predefined engagement position.

44. The device of claim 37, wherein the at least one holder is adapted for back and forth movement between the non-engagement position and the predefined engagement position via at least one predefined intermediate position located between the non-engagement position and the predefined engagement position, and wherein a latching device is provided to hold the at least one holder in the predefined intermediate position.

45. The device of claim 37, wherein the at least one holder is adapted to move the element to be brought into engagement with said eye of the user into the predefined engagement position by a rotary movement, and wherein an axis of rotation for the rotary movement is a vertical axis or a horizontal axis, the horizontal axis lying in a vertical direction above said eye of the user.

46. The device of claims 45, wherein the at least one holder has an actuating handle for moving the at least one holder between the non-engagement position and the predefined engagement position, and wherein at least one of the actuating handle is arranged in an axial direction along the axis of rotation, the actuating handle protrudes in a horizontal direction beyond the spectacle-like frame, and said device is adapted such that the actuating handle is arranged outside a field of view of the user.

47. The device of claim 37, wherein the at least one holder is adapted for back and forth movement between the non-engagement position and the predefined engagement position single-handedly and without tools.

48. The device of claim 37, wherein the at least one holder is adapted to bring the element to be brought into engagement with said eye of the user into the predefined engagement position from a front of said eye of the user at an angle in the range of 30 to the horizontal.

49. The device according to claim 46, further comprising:
an operating element arranged in the actuating handle for moving the at least one holder in a horizontal direction relative to the spectacle-like frame.

50. The device of claim 37, wherein the at least one holder includes a nasal vertical arm and a temporal vertical arm which extend downward in a vertical direction from the at least one holder from a horizontal crossbar of the at least one holder, and wherein the nasal vertical arm and the temporal vertical arm include adjustable lengths.

51. The device of claim 50, wherein the at leas one holder includes a nasal horizontal leg and a temporal horizontal leg, he nasal horizontal leg comprising a first receptacle for the element to be brought into engagement with said eye of the user, and the temporal horizontal leg comprising a second receptacle for the element to be brought into engagement with said eye of the user, and wherein the nasal horizontal leg and the temporal horizontal leg include adjustable lengths.

52. The device of claim 51, wherein the horizontal crossbar is arranged at the spectacle-like frame and adjustable in a horizontal direction, and wherein the nasal horizontal leg is arranged at a lower end of the nasal vertical arm and the temporal horizontal leg is arranged at a lower end of the temporal vertical arm.

53. The device of claim 37, wherein the at least one holder is adapted to bring the element into engagement with said eye of the user at a first support point and a second support point into engagement with said eye of the user in the predefined engagement position, and wherein the at least one holder is adapted to adjust the first support point in three spatial directions and to adjust the second support point in three spatial directions, wherein exactly one spatial direction is coupled such that the first and second support points have a fixed distance in this spatial direction.

54. The device of claim 37, wherein the element to be brought into engagement with said eye of the user is a contact lens, and wherein the at least one holder is a holder for the contact lens and is adapted to apply the contact lens to said eye of the user in the predefined engagement position.

55. A system for electrostimulation of said eye comprising said device according to claim 37 and a stimulation electrode arrangement for electrostimulation of said eye, wherein the element to be brought into engagement with said eye of the user is a stimulation electrode and wherein the holder is an electrode holder for the stimulation electrode.

56. A method, in particular a method for applying a stimulation electrode of a device for electrostimulation of said eye to said eye of the user, wherein the method comprises the following steps:
a) providing said device according to claim 37;
b) placing said device on the head of the user, wherein the holder is in the non-engagement position; and
c) moving the holder from the non-engagement position to the predefined engagement position after placing said device on the head of the user, to bring the element to be brought into engagement with said eye of the user into engagement with said eye of the user.
